(19) United States
(12) Patent Application Publication
(10) Pub. No.: US 2022/0193341 A1
(43) Pub. Date: Jun. 23, 2022

(54) SYRINGE SPLITTING DEVICE

(71) Applicant: University of Kansas, Lawrence, KS (US)

(72) Inventors: Shikhar Tomur, Kansas City, MO (US); Christopher Tomassian, Kansas City, MO (US)

(21) Appl. No.: 17/554,676

(22) Filed: Dec. 17, 2021

Related U.S. Application Data
(60) Provisional application No. 63/129,062, filed on Dec. 22, 2020.

(51) Int. Cl. A61M 5/178; A61M 5/168; A61M 39/10

(52) U.S. Cl. CPC A61M 5/1782; A61M 5/16827; A61M 2005/3114; A61M 39/1011; A61M 39/105

(57) ABSTRACT

Various embodiments of a device for splitting fluid substances and methods thereof are described. In one example, a device for splitting fluid substances among syringes includes an input port, a number of output ports, and a fluid distribution network. The fluid distribution network is formed to convey a fluid substance from the input port to the output ports. The fluid distribution network can include a number of fluid distribution channels for conveying the fluid substance from the input port to the output ports. The fluid distribution channels can be formed in a star configuration, in one example, although other configurations are possible. The device provides a more efficient way to fill smaller syringes during procedures, such as fat grafting procedures. The device can be used to fill a number of syringes concurrently, decreasing the time required to perform an operation, among other benefits.

### CROSS-REFERENCE TO RELATED APPLICATIONS

This application claims the benefit of and priority to U.S. Provisional Application No. 63/129,062, filed Dec. 22, 2020, the entire disclosure of which is hereby incorporated herein by reference.

### BACKGROUND

Transferring, grafting, or injecting fat is a surgical process by which fat can be transferred from one area of the body to another. The object of the procedure is to augment the area where the fat is injected. The procedure involves extracting adipose fat by liposuction, processing the fat, and then reinjecting the fat into the area for augmentation. Surgeons have used fat grafting to enhance the cosmetic appearance of certain areas, such as the face, breast, and buttocks. Fat grafting has been shown to have other benefits however, including for healing wounds, diminishing scars, and repairing tissue following radiation and other treatments.

The fat grafting procedure generally involves three steps, including the extraction of fat from a donor area with liposuction, the decanting, centrifugation, and processing of the fat, and the reinjection of the purified fat into the area for treatment. Fat extraction is often performed manually from a first body area by a surgeon using liposuction cannulas. The fat is then processed to separate excess fluid, debris, and unwanted or undesirable cells. The fat is then reinjected into the subcutaneous tissue of the patient at a second body area. The amount or volume of fat being reinjected is typically measured in cubic centimeters or milliliters and varies based on various factors, including the type of procedure being performed and the area of the body where the fat is being injected.

### SUMMARY OF THE INVENTION

Various embodiments of a device for splitting fluid substances and methods thereof are described. In one example, a device for splitting fluid substances among syringes includes an input port, a plurality of output ports, and a fluid distribution network. The fluid distribution network is formed to convey a fluid substance from the input port to the plurality of output ports. The fluid distribution network can include a number of fluid distribution channels for conveying the fluid substance from the input port to the plurality of output ports. The fluid distribution channels can be formed in a star configuration, in one example, although other configurations are possible. The device provides a more efficient way to fill smaller syringes during procedures, such as fat grafting procedures. The device can be used to fill a number of syringes concurrently, decreasing the time required to perform an operation, among other benefits.

In one aspect of the embodiments, a device for splitting fluid substances includes an upper body component and a lower body component. The input port and the number of output ports are integrally formed in the upper body component. The fluid distribution network is integrally formed in the lower body component. The upper body component and the lower body component are sealingly secured together.

In other aspects of the embodiments, the fluid distribution network includes a number of radially-extending fluid distribution channels integrally formed in the lower body component for conveying the fluid substance from the input port to the plurality of output ports. The radially-extending fluid distribution channels are formed in a star configuration in one example. In this case, each output port is in fluid communication with a respective one of a number of radially-extending fluid distribution channels in the network. In other examples, the fluid distribution network is formed in a leaf configuration, an alternate leaf configuration, or a spoke and semi-circle configuration. Each output port among the output ports is in fluid communication with the fluid distribution network among the examples. In various examples, the fluid distribution network can include a fluid channel in a series arrangement between the input port and the plurality of output ports or a fluid channel in a parallel arrangement between the input port and the plurality of output ports.

In other aspects of the embodiments, the input port includes a press-fit connector. In another example, the input port includes a luer lock connector. The input port can also include a press-fit connector with an interference-fit ledge stop. The input port can also include a tapered port leading to the fluid distribution network.

A method, process, or procedure for splitting fluid substances among syringes is also described. The procedure includes fitting a larger syringe onto an input port of a device for splitting a fluid substance, depressing a plunger of the larger syringe until the fluid substance approaches at least one of a plurality of output ports of the device, securing smaller syringes onto the plurality of output ports of the device, and further depressing the plunger of the larger syringe to fill the smaller syringes with the fluid substance. The procedure can also include removing the smaller syringes from the device and discarding the device.

### BRIEF DESCRIPTION OF THE DRAWINGS

For a more complete understanding of the embodiments and the advantages thereof, reference is now made to the following description, in conjunction with the accompanying figures briefly described as follows:

FIG. 1 illustrates an example device for splitting fluid substances according to various embodiments described herein.

FIG. 2 illustrates a top-down view of the device for splitting fluid substances shown in FIG. 1 according to various embodiments described herein.

FIG. 3 illustrates a side view of the device for splitting fluid substances shown in FIG. 1 according to various embodiments described herein.

FIG. 4 illustrates a bottom view of the device for splitting fluid substances shown in FIG. 1 according to various embodiments described herein.

FIG. 5 illustrates a lower body component of the device for splitting fluid substances shown in FIG. 1 according to various embodiments described herein.

FIG. 6 illustrates another example device for splitting fluid substances according to various embodiments described herein.

FIG. 7 illustrates a bottom view of the device shown in FIG. 6 according to various embodiments described herein.

FIG. 8 illustrates a lower body component of the device shown in FIG. 6 according to various embodiments described herein.

FIG. 9 illustrates a bottom view of another lower body component according to various embodiments described herein.

FIG. 10 illustrates a bottom view of the device shown in FIG. 6, with a different lower body component, according to various embodiments described herein.

FIG. 11 illustrates the lower body component of the device shown in FIG. 10 according to various embodiments described herein.

FIG. 12A illustrates another example device for splitting fluid substances according to various embodiments described herein.

FIG. 12B illustrates a cross-sectional view of the device for splitting fluid substances shown in FIG. 12A according to various embodiments described herein.

FIG. 13 illustrates a cross-sectional view of another device for splitting fluid substances according to various embodiments described herein.

FIG. 14 illustrates an example process for a fluid splitting procedure according to various embodiments described herein.

The drawings illustrate only example embodiments and are therefore not to be considered limiting of the scope of the embodiments described herein, as other embodiments are within the scope of this disclosure. The elements and features shown in the drawings are not necessarily drawn to scale, emphasis instead being placed upon clearly illustrating the principles of the embodiments. Additionally, certain dimensions or positionings may be exaggerated to help visually convey certain principles. In the drawings, similar reference numerals between figures designate like or corresponding, but not necessarily the same, elements.

### DETAILED DESCRIPTION

As noted above, the fat grafting procedure generally involves extracting fat from a first area, processing the fat, and reinjecting the fat into a second area for treatment. The amount or volume of fat being extracted and reinjected is typically measured in cubic centimeters or milliliters and varies based on the procedure being performed and other factors.

One issue facing surgeons during fat grafting procedures is the need to easily transfer fat from one type of vessel, container, or canister to other vessels or containers. As one example, adipose fat can be extracted from the body and collected into a container or canister, such as the container of the REVOLVE™ Advanced Adipose System. The fat can be processed, either within or outside the container, to separate excess fluid, debris, and unwanted cells from the fat for reinjection. The container can store 30-80 cubic centimeters (cc) or milliliters (ml) of fluid, or more, for example.

From the container, the fat must be transferred into syringes suitable for reinjection. Fat for reinjection can be transferred into 10 cc syringes, 5 cc syringes, or 1 cc syringes, as examples. One approach to transfer the fat from a larger container to syringes involves the use of one or more intermediary syringes, such as the 60 cc Toomey syringe, or other syringes of relatively large volume or size. The intermediary syringe can be relied upon to draw fat from the larger container. The fat in the intermediary syringe can then be transferred, in steps, into a number of smaller syringes, such as 10 cc or 5 cc syringes, for reinjection. Depending upon the size of the intermediate syringe, it can be used to fill several smaller syringes. The transfer from the intermediate syringe to the smaller syringes can be facilitated through the use of a luer lock-to-luer lock adapter.

The transfer of fluid substances, including fat, from an intermediate syringe to several smaller syringes can be time consuming. Also, the luer lock-to-luer lock adapter can be cumbersome to use. Some of the fluid substance can be lost during the transfer. Moreover, because the adapter may be formed from stainless steel or other reusable materials, it can be cleaned and sterilized between uses. However, it can be relatively difficult to clean all the internal and external surfaces of the reusable adapters. Thus, stray cells, including bacteria, can remain hidden in these adapters, increasing the chances for the spread of infections during procedures.

In the context outlined above, the embodiments described herein are directed to a device for splitting fluid substances among syringes and methods thereof. In one example, a device for splitting fluid substances among syringes includes an input port, a number of output ports, and a fluid distribution network. The distribution network is formed to convey a fluid substance from the input port to the output ports. The fluid distribution network can include a number of fluid distribution channels for conveying the fluid substance from the input port to the output ports. The fluid distribution channels can be formed in a star configuration, in one example, although other configurations are possible. The device provides a more efficient way to fill smaller syringes during procedures, such as fat grafting procedures. The device can be used to fill a number of syringes concurrently, decreasing the time required to perform an operation, among other benefits.

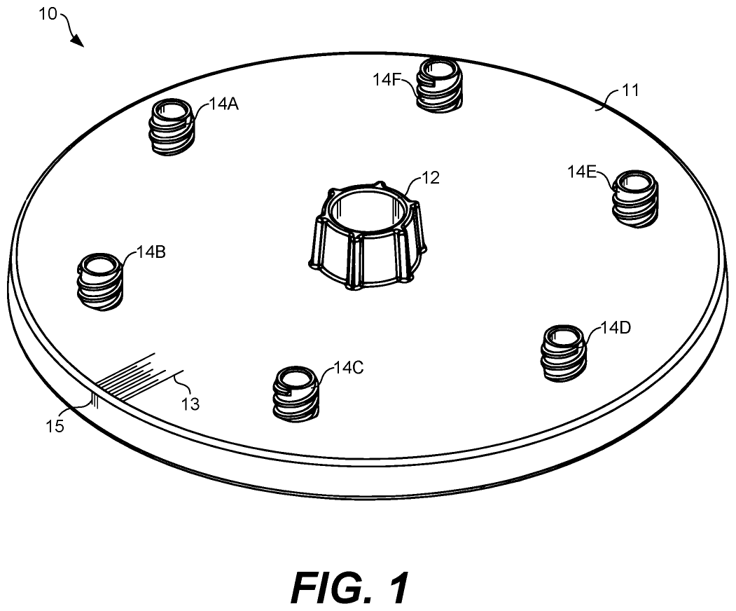

Turning to the drawings, FIG. 1 illustrates an example device 10. The device 10 can be used for splitting, dividing, and transferring fluid substances among syringes. The device 10 is provided as a representative example device for splitting fluid substances among syringes according to the concepts described herein. Other devices incorporating the concepts can vary in size, shape, number of ports, and other features as compared to the device 10.

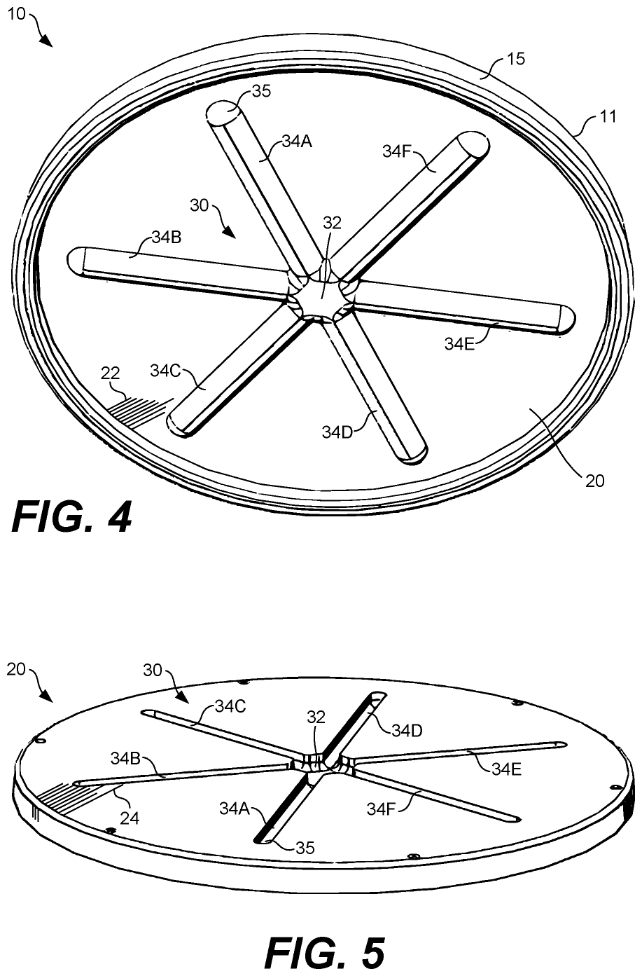

As shown in FIG. 1, the device 10 includes an upper body component 11. The upper body component 11 includes an input port 12 and a plurality of output ports 14A-14F (collectively, “output ports 14”) that extend up from a top surface 13 of the upper body component 11. The input port 12 includes an aperture (e.g., a circular opening as shown) that extends through the upper body component 11, and each of the output ports 14 includes an aperture that extends through the upper body component 11. The upper body component 11 also includes a side surface 15, among other surfaces not shown in FIG. 1. The device 10 also includes a lower body component which is illustrated in FIGS. 4 and 5 and described in further detail below. In other cases, the lower body component of the device 10 can be embodied as the examples shown in FIGS. 4, 5, 7-9, 10, and 11.

The device 10, and the related devices 100, 200, and 300 described herein, can vary in certain aspects as compared to that shown. a plurality of output ports 14A-14F (collectively, “output ports 14”) that extend up from a top surface 13 of the upper body component 11. The input port 12 includes an aperture (e.g., a circular opening as shown) that extends through the upper body component 11, and each of the output ports 14 includes an aperture that extends through the upper body component 11. The upper body component 11 also includes a side surface 15, among other surfaces not shown in FIG. 1. The device 10 also includes a lower body component which is illustrated in FIGS. 4 and 5 and described in further detail below. In other cases, the lower body component of the device 10 can be embodied as the examples shown in FIGS. 4, 5, 7-9, 10, and 11.

In one example, the upper body component 11 can range in size from about 6 inches to about 12 inches or more in diameter, although smaller and larger sizes are within the scope of the embodiments. The upper body component 11 is illustrated in a circular shape in FIG. 1. The upper body component 11 and the device 10 can be formed in other shapes, however, such as triangular, oval, rectangular, square, hexagon, octagon, etc.

The input port 12 is relatively larger in size than the output ports 14, to allow for the input of a relatively larger volume of fluid substances, as compared to the relatively smaller volume of fluid substances output at the output ports 14. In the example shown in FIG. 1, the input port 12 is embodied as a soft-tip syringe press-fit connector, and the open end of a relatively large syringe can be press-fit into the input port 12. Each of the output ports 14 includes a luer lock connector, and the open end of syringes can be secured onto the output ports 14 using the luer lock connectors. In alternative embodiments, the input port 12 and the output ports 14 can include other types or styles of connectors for mechanical attachment to syringes.

The device 10 also includes a fluid distribution network, although it is not visible in FIG. 1. The fluid distribution network is integrally formed in the lower body component, as described in further detail below. The fluid distribution network is formed to convey a fluid substance from the input port 12 to the output ports 14. Thus, if a syringe of larger volume is coupled at the input port 12, the contents of the syringe can be expelled through the input port 12, into the device 10, and routed to the output ports 14. If syringes of relatively smaller volume are coupled at the output ports 14, the contents of the larger syringe at the input port 12 can be transferred into the smaller syringes at the output ports 14. Aspects of the fluid distribution network are described in further detail below with reference to FIGS. 4 and 5.

The device 10 offers a number of advantages in the transfer of fluid substances, including fat, from larger syringes to smaller syringes. Use of the device 10 reduces the chance of fluid substance loss and reduces the time needed to transfer the fluid substance. Also, the device 10 can be formed from plastic or other disposable materials. Thus, the device 10 can be discarded with each procedure, reducing (or eliminating) the chances for bacteria or other contaminants being distributed during the fluid transfer procedure.

In one embodiment, the device 10 is formed as a disposable, one-time-use device. In that case, the device 10 may be discarded after being used in a medical procedure. Alternatively, the device 10 can be used in a number of different procedures and cleaned and sterilized between procedures. The device 10 can also be formed to any suitable size based on various factors, including the type(s) and/or size(s) of syringes being connected to it, the number of input ports, output ports, and/or other factors.

The device 10 can be formed from any suitable type(s) of inert materials, including plastic(s), metal(s), rubber(s), ceramic(s), glass, other materials, and combinations thereof. In one preferred embodiment, the device 10 can be formed from a disposable plastic, such as polycarbonate, polypropylene, or nylon. In some cases, one or more parts of the device 10 can be formed from flexible material(s) while other parts are formed from more rigid materials. For example, the upper body component 11 can be formed from rigid materials, and the lower body component (FIGS. 4 & 5) can be formed from flexible materials.

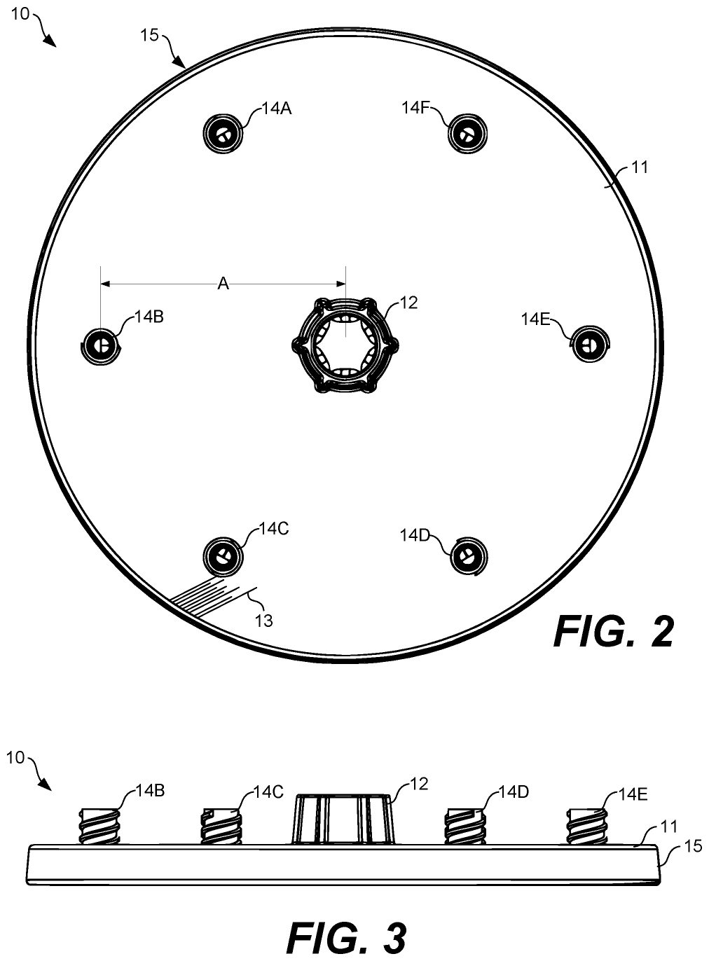

Turning to other views, FIG. 2 illustrates a top-down view of the device 10. As shown in FIG. 2, the input port 12 is centrally located or positioned on the upper body component 11. The output ports 14 are equally spaced-apart from each other, and each output port 14A-14F is positioned a radial distance “A” apart from a center of the input port 12. The radial distance “A” can vary among the embodiments. The number of output ports 14 can also vary among the embodiments, and the device 10 can include fewer or more output ports than the six (6) shown in FIG. 2. FIG. 3 illustrates a side view of the device 10. As shown, the input port 12 is relatively wider and taller than each of the output ports 14.

FIG. 4 illustrates a bottom view of the device 10 according to various embodiments described herein. The bottom surface 22 of the lower body component 20 of the device 10 is visible in FIG. 4. The lower body component 20 includes the fluid distribution network 30, which is described below. The device 10 is formed from the upper body component 11 and the lower body component 20, which can be press-fitted together. The upper body component 11 and the lower body component 20 can also be fused together, using adhesives, chemical, sonic, heat welds, or other suitable means. In any case, the upper body component 11 and the lower body component 20 can be sealingly secured together, to prevent fluid substances from escaping the fluid distribution network 30.

As shown, the lower body component 20 includes the fluid distribution network 30, which is formed in a star configuration as illustrated. The fluid distribution network 30 includes an open passageway or channel between the input port 12 and each of the output ports 14. The fluid distribution network 30 includes a central distribution hub 32 and a number of fluid distribution channels 34A-34F (collectively, “distribution channels 34”) for conveying fluid substances from the input port 12 to a respective one of output ports 14. A channel bend 35 is included at one end of the distribution channel 34A, and the distribution channels 34B-34F also include similar channel bends.

FIG. 5 illustrates a top view of the lower body component 20, separate from of the device 10 according to various embodiments described herein. It is clear from FIG. 5 that the central distribution hub 32 and fluid distribution channels 34A-34F are formed as depressed channels in the top surface 24 of the lower body component 20. The depressed channels are capable of conveying fluid substances from the input port 12 to the output ports 14. When assembled with the upper body component 11, the distal ends (i.e., most distant from the central distribution hub 32) of the fluid distribution channels 34A-34F terminate or end just below the apertures or openings of the output ports 14A-14F. Thus, a fluid substance pushed or pressed with sufficient force into the input port 12 will enter the central distribution hub 32, spread and distribute among the fluid distribution channels 34A-34F, flow radially through the fluid distribution channels 34A-34F, and flow up and into the output ports 14A-14F. When syringes are coupled to the output ports 14A-14F, the fluid substance will flow into the syringes, filling the syringes.

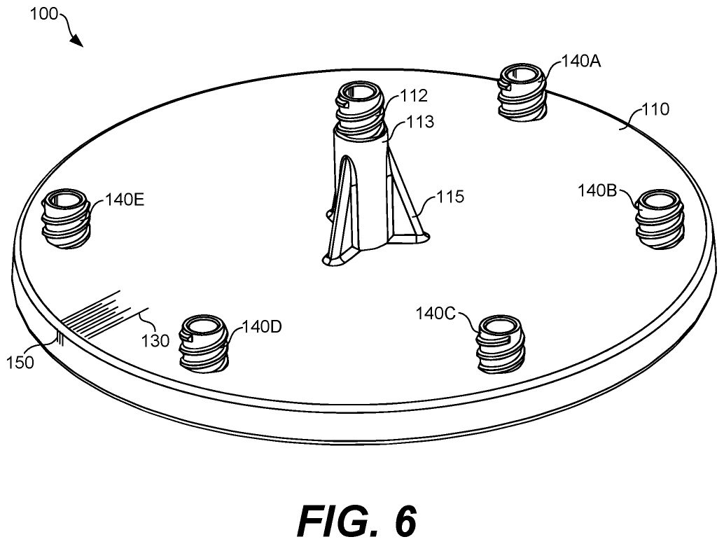

FIG. 6 illustrates another example device 100 for splitting fluid substances according to various embodiments described herein. The device 100 can be used for splitting, dividing, and transferring fluid substances among syringes. The device 100 is provided as a representative example, and the device 100 can vary in size, shape, number of ports, and other features.

The device 100 includes an upper body component 110 and a lower body component. The lower body component is not visible in FIG. 6, but example lower body components that can be relied upon in the device 100 are shown in FIGS. 4, 5, 7-9, 10, and 11. The upper body component 110 includes an input port 112 and a plurality of output ports 140A-140E (collectively, “output ports 140”) that extend up from a top surface 130 of the upper body component 110. The input port 112 includes an aperture (e.g., a circular opening as shown) that extends through the upper body component 110, and each of the output ports 140 includes an aperture that extends through the upper body component 110. The upper body component 110 also includes a side surface 150, among other surfaces. The device 100 also includes a lower body component, although it is not visible in FIG. 6.

In the example shown in FIG. 6, the input port 112 is similar in size (e.g., in inner diameter) as compared to each of the output ports 140, although the input port 112 can be larger than the output ports 140 in some cases. The input port 112 and each of the output ports 140 includes as a luer lock connector. Syringes can be secured onto the input port 112 and each of the output ports 140 using the luer lock connectors. In alternative embodiments, the input port 112 and the output ports 140 can include other types or styles of connectors.

The upper body component 110 also includes an extension pedestal 113, and the input port 112 is positioned at the distal end of the extension pedestal 113. The extension pedestal 113 includes an internal cylindrical fluid channel, leading from the open end of the input port 112 to the fluid distribution network in the lower body component. A number of braces 115 are integrally formed in the upper body component 110. The braces 115 extend between the top surface 130 of the upper body component 110 and the outer side surface of the extension pedestal 113, to add structural support to the extension pedestal 113. The extension pedestal 113 helps to raise or extend the input port 112 up and away from the top surface 130 of the upper body component 110. In that way, the extension pedestal 113 can help to avoid any mechanical interference between syringes coupled to the input port 112 and syringes coupled to the output ports 140, particularly when the device 100 is in use.

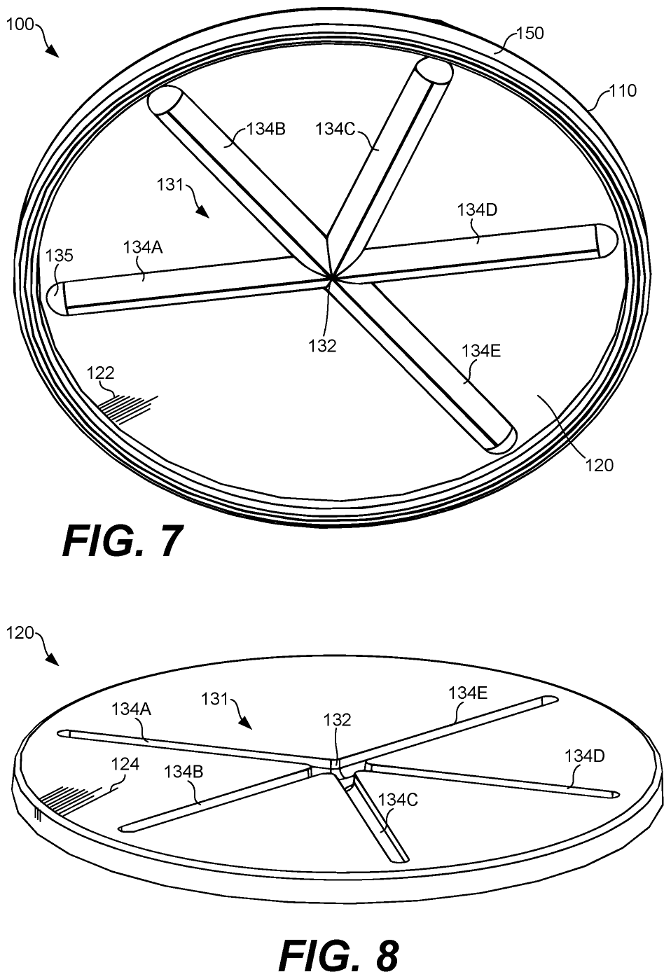

The device 100 also includes a fluid distribution network within the device 100. The fluid distribution network is formed to convey a fluid substance from the input port 112 to the output ports 140. Thus, if a syringe of larger volume is coupled at the input port 112, the contents of the syringe can be expelled through the input port 112, into the device 100, and routed to the output ports 140. If syringes of relatively smaller volume are coupled at the output ports 140, then the contents of the larger syringe at the input port 112 can be transferred into the smaller syringes at the output ports 140. Aspects of the fluid distribution network are described in further detail below with reference to FIGS. 7 and 8.

The device 100 offers a number of advantages in the transfer of fluid substances, including fat, from larger syringes to smaller syringes. Use of the device 100 reduces the chance of fluid substance loss and reduces the time needed to transfer the fluid substance. Also, the device 100 can be formed from plastic or other disposable materials. Thus, the device 100 can be discarded with each procedure, reducing (or eliminating) the chances for bacteria or other contaminants being distributed during the fluid transfer procedure.

In one embodiment, the device 100 is formed as a disposable, one-time-use device. In that case, the device 100 may be discarded after being used in a medical procedure. Alternatively, the device 100 can be used in a number of different procedures and cleaned and sterilized between procedures. The device 100 can also be formed to any suitable size based on various factors, including the type(s) and/or size(s) of syringes being connected to it, the number of input ports, output ports, and/or other factors.

Like the device 10 shown in FIGS. 1-3, the device 100 can be formed from any suitable type(s) of inert materials, including plastic(s), metal(s), rubber(s), ceramic(s), glass, other materials, and combinations thereof. In one preferred embodiment, the device 100 can be formed from a disposable plastic, such as polycarbonate, polypropylene, or nylon. In some cases, one or more parts of the device 100 can be formed from flexible material(s) while other parts are formed from more rigid materials. For example, the upper body component 110 can be formed from rigid materials, and the lower body component can be formed from flexible materials.

The device 100 is similar to the device 10 in FIG. 1. However, there are a number of differences between the device 100 and the device 10. For example, the device 100 includes five output ports 140A-140E rather than the six output ports 14A-14F of the device 10. Additionally, the device 100 includes a luer lock connector at the end of the input port 112 rather than the soft-tip syringe press-fit connector of the input port 12 of the device 10. In other cases, the device 100 can omit one or more of the output ports 140A-140E, include additional output ports similar to the output ports 140A-140E, include a soft-tip syringe press-fit connector in place of the luer lock connector at the end of the input port 112, or include other features described among the embodiments described herein.

FIG. 7 illustrates a bottom view of the device 100 according to various embodiments described herein. The bottom surface 122 of the lower body component 120 of the device 100 is visible in FIG. 7. The lower body component 120 includes the fluid distribution network 131, which is described below. The device 100 is formed from the upper body component 110 and the lower body component 120, which can be press-fitted together. The upper body component 110 and the lower body component 120 can also be fused together, using adhesives, chemical, sonic, heat welds, or other suitable means. The upper body component 110 and the lower body component 120 can be sealingly secured together, to prevent fluid substances from escaping the fluid distribution network 131.

As shown, the lower body component 120 includes the fluid distribution network 131, which is formed in a leaf configuration as shown. The fluid distribution network 131 includes an open passageway or channel between the input port 112 and each of the output ports 140. The fluid distribution network 131 includes a central distribution hub 132 and a number of fluid distribution channels 134A-134E (collectively, “distribution channels 134”) for conveying fluid substances from the input port 112 to a respective one of the output ports 140. A channel bend 135 is included at one end of the distribution channel 134A, and each of the distribution channels 134B-134F also includes a similar channel bend. The channel bends help to direct fluid up towards the output ports 140.

FIG. 8 illustrates a top view of the lower body component 120, separate from the device 100. It is clear from FIG. 8 that the central distribution hub 132 and fluid distribution channels 134A-134E are formed as depressed channels in the top surface 124 of the lower body component 120. The depressed channels are capable of conveying fluid substances from the input port 112 to the output ports 140. When assembled with the upper body component 110, the distal ends (i.e., most distant from the central distribution hub 132) of the fluid distribution channels 134A-134E terminate or end just below the apertures or openings of the output ports 140A-140E. Thus, a fluid substance pushed or pressed with sufficient force into the input port 112 will enter the central distribution hub 132, spread and distribute among the fluid distribution channels 134A-134E, flow radially through the fluid distribution channels 134A-134F, and flow up and into the output ports 140A-140E. When syringes are coupled to the output ports 140A-140E, the fluid substance will flow into the syringes, filling the syringes.

The lower body component 120 is similar to the lower body component 20 in FIG. 5. However, there are a number of differences between the lower body component 120 and the lower body component 20. For example, the lower body component 120 includes five radially-extending fluid distribution channels 134A-134E rather than the six radially-extending fluid distribution channels 34A-34F of the lower body component 20. In other cases, the lower body component 120 can include fewer than five radially-extending fluid distribution channels, such as four, three, or two channels. In other cases, the lower body component 120 can include more than five or six radially-extending fluid distribution channels, such as seven, eight, or more channels. The channels can be evenly distributed apart from each other, such as how the radially-extending fluid distribution channels 34A-34F are evenly spaced apart. Alternatively, the channels can be unevenly distributed apart from each other, such as how the radially-extending fluid distribution channels 134A-134E are not evenly spaced apart.

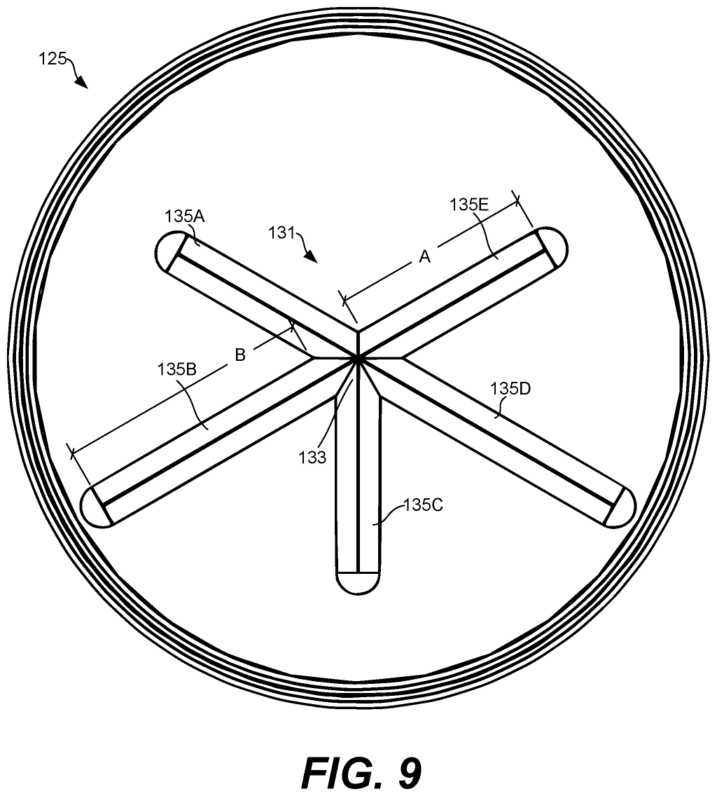

In FIGS. 4 and 5, the fluid distribution network 30 is formed in a star configuration in the lower body component 20. In FIGS. 7 and 8, the fluid distribution network 131 is formed in a leaf configuration in the lower body component 120. However, other types, arrangements, or styles of fluid distribution networks are within the scope of the embodiments. As one example, FIG. 9 illustrates a bottom view of another lower body component 125 including the fluid distribution network 131. The fluid distribution network 131 is formed in an alternate leaf configuration, with fluid distribution channels having different lengths. The lower body component 125 can be used in any of the devices for splitting fluid substances described herein.

As shown in FIG. 9, the fluid distribution network 131 includes a central distribution hub 133 and the fluid distribution channels 135A-135E (collectively, “distribution channels 135”) for conveying fluid substances from an input port to output ports. In the fluid distribution network 131, the fluid distribution channel 135E extends radially to a distance “A” away from the central distribution hub 133 and the fluid distribution channel 135B extends radially to a distance “B” away from the central distribution hub 132, where “A” is shorter than “B”. Thus, the fluid distribution network 131 is an example having fluid distribution channels 135A-135E of various, different radially-extending, lengths.

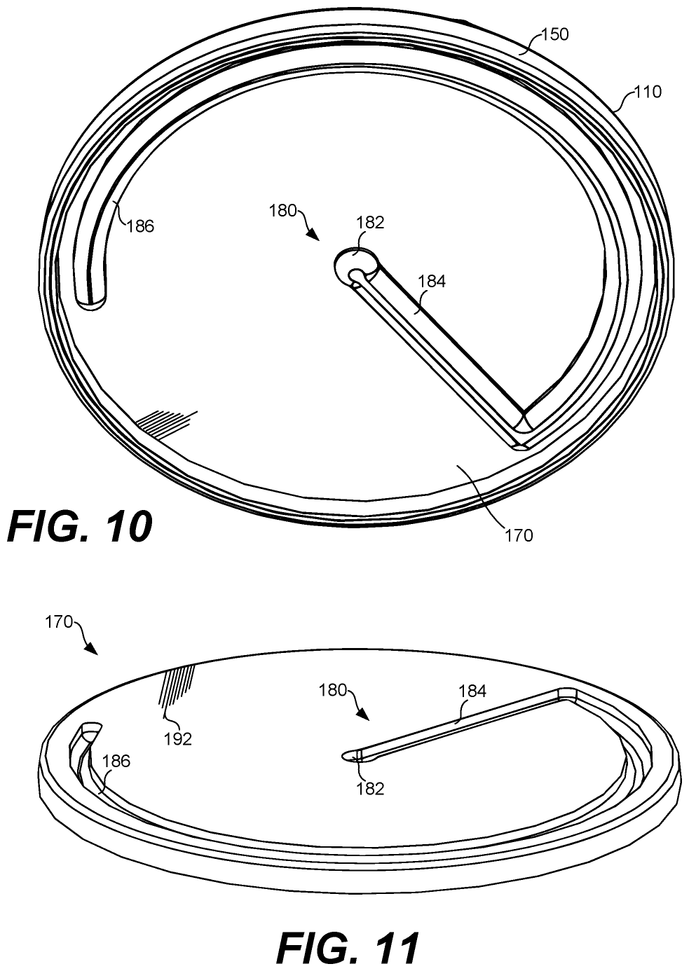

In other examples, FIG. 10 illustrates a bottom view of the device 100 shown in FIG. 6, with a different lower body component 170, and FIG. 11 illustrates a top view of the lower body component 170. The lower body component 170 includes a fluid distribution network 180 integrally formed in the lower body component 170 for conveying the fluid substance from the input port 112 to the output ports 140. The fluid distribution network 180 is formed in a spoke and semi-circle configuration and includes a central distribution hub 182, a radially-extending fluid distribution channel 184, and a circularly-extending fluid distribution channel 186.

It is clear from FIG. 11 that fluid distribution network 180 is formed as depressed channels in the top surface 192 of the lower body component 170. The depressed channels are capable of conveying fluid substances from the input port 112 to the output ports 140. When assembled with the upper body component 110, the circularly-extending fluid distribution channel 186 extends below the apertures or openings of the output ports 140A-140E. Thus, a fluid substance pushed or pressed with sufficient force into the input port 112 will enter the central distribution hub 182, flow along the radially-extending fluid distribution channel 184, flow and spread into the circularly-extending fluid distribution channel 186, and flow up and into the output ports 140A-140E. When syringes are coupled to the output ports 140A-140E, the fluid substance will flow into the syringes, filling the syringes.

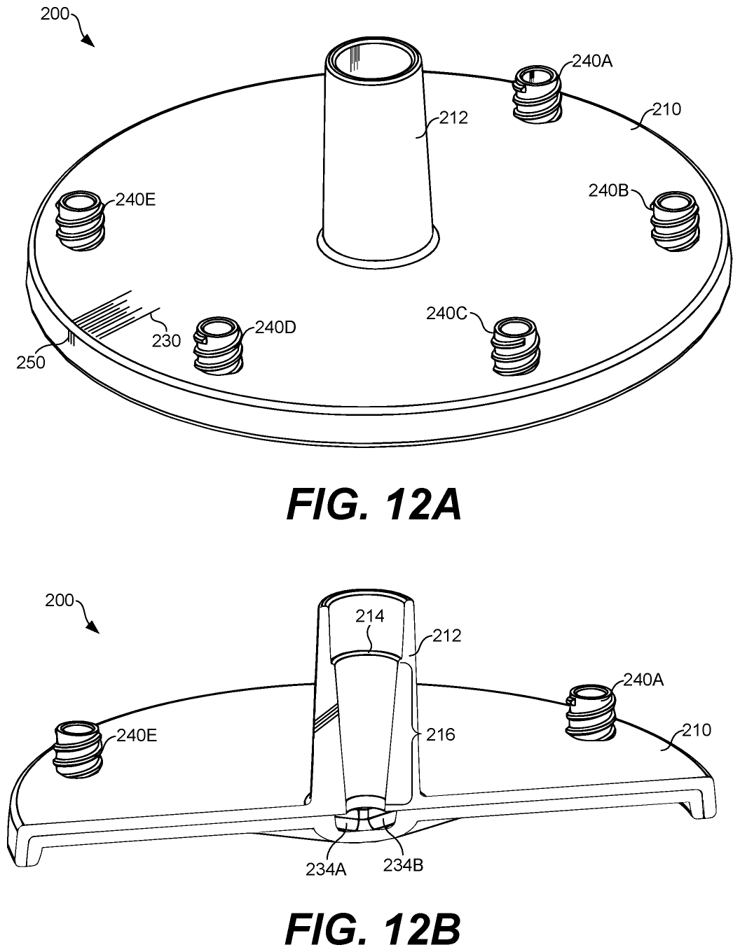

FIG. 12A illustrates another example device 200 for splitting fluid substances according to various embodiments described herein. The device 200 can be used for splitting, dividing, and transferring fluid substances among syringes. The device 200 is provided as a representative example device for splitting fluid substances among syringes according to the concepts described herein. Other devices incorporating the concepts can vary in size, shape, number of ports, and other features as compared to the device 200.

The device 200 includes an upper body component 210. The upper body component 210 includes an input port 212 and a number of output ports 240A-240E (collectively, “output ports 240”) that extend up from a top surface 230 of the upper body component 210. The input port 212 includes an aperture (e.g., a circular opening as shown) that extends through the upper body component 210, and each of the output ports 240 includes an aperture that extends through the upper body component 210. The upper body component 210 also includes a side surface 250, among other surfaces. The device 200 also includes a lower body component, although it is not visible in FIG. 12A. The lower body component of the device 200 can be embodied as any of the examples shown in FIGS. 4, 5, 7-9, 10, and 11, among related lower body components.

The input port 212 is relatively larger in size than the output ports 240, to allow for the input of a relatively larger volume of fluid substances, as compared to the relatively smaller volume of fluid substances output at the output ports 240. In the example shown in FIG. 12A, the input port 212 is embodied as a raised soft-tip syringe press-fit connector, and the open end of a relatively large syringe can be press-fit into the input port 212. Each of the output ports 240 includes a luer lock connector, and the open end of syringes can be secured onto the output ports 240 using the luer lock connectors. In alternative embodiments, the input port 212 and the output ports 240 can include other types or styles of connectors for mechanical attachment to syringes.

The device 200 also includes a fluid distribution network, although it is not visible in FIG. 12A. The fluid distribution network is integrally formed in the lower body component of the device 200, as described herein. The fluid distribution network is formed to convey a fluid substance from the input port 212 to the output ports 240. Thus, if a syringe of larger volume is coupled at the input port 212, the contents of the syringe can be expelled into and through the input port 212, into the device 200, and routed to the output ports 240. If syringes of relatively smaller volume are coupled at the output ports 240, the contents of the larger syringe at the input port 212 can be transferred into the smaller syringes at the output ports 240.

Like the other fluid splitting devices described herein, the device 200 offers a number of advantages in the transfer of fluid substances, including fat, from larger syringes to smaller syringes. Use of the device 200 reduces the chance of fluid substance loss and reduces the time needed to transfer the fluid substance. Also, the device 200 can be formed from plastic or other disposable materials. Thus, the device 200 can be discarded with each procedure, reducing (or eliminating) the chances for bacteria or other contaminants being distributed during the fluid transfer procedure.

In one embodiment, the device 200 is formed as a disposable, one-time-use device. In that case, the device 200 may be discarded after being used in a medical procedure. Alternatively, the device 200 can be used in a number of different procedures and cleaned and sterilized between procedures. The device 200 can also be formed to any suitable size based on various factors, including the type(s) and/or size(s) of syringes being connected to it, the number of input ports, output ports, and/or other factors.

The device 200 can be formed from any suitable type(s) of inert materials, including plastic(s), metal(s), rubber(s), ceramic(s), glass, other materials, and combinations thereof. In one preferred embodiment, the device 200 can be formed from a disposable plastic, such as polycarbonate, polypropylene, or nylon. In some cases, one or more parts of the device 200 can be formed from flexible material(s) while other parts are formed from more rigid materials. For example, the upper body component 210 can be formed from rigid materials, and the lower body component can be formed from flexible materials.

FIG. 12B illustrates a cross-sectional view of the device 200 for splitting fluid substances. A cross-section of the input port 212 is shown in FIG. 12B. The input port 212 includes a press-fit connector with an interference-fit ledge stop 214 and a tapered port 216 leading to the fluid distribution network of the device 200. The interference-fit ledge stop 214 provides a mechanical interference and stop for a soft-tip press-fit connector of a syringe to be seated upon. The tapered port 216 tapers more narrowly as it extends from the interference-fit ledge stop 214 to the fluid distribution network, which includes the fluid distribution channels 234A and 234B, among other channels.

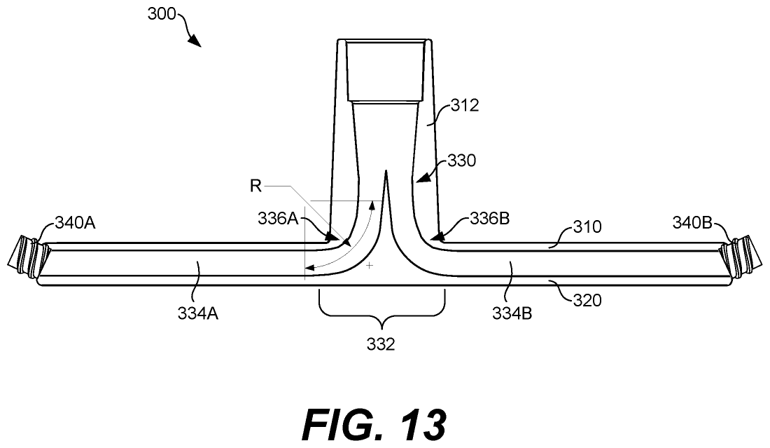

FIG. 13 illustrates a cross-sectional view of another device 300 for splitting fluid substances according to various embodiments described herein. The device 300 can be used for splitting, dividing, and transferring fluid substances among syringes. The device 300 is provided as a representative example device for splitting fluid substances among syringes according to the concepts described herein. Other devices incorporating the concepts can vary in size, shape, number of ports, and other features as compared to the device 300.

The device 300 includes an upper body component 310. The upper body component 310 includes an input port 312 and output ports 340A and 340B, among others. The input port 312 includes an aperture (e.g., a circular opening as shown) that extends through the upper body component 310. The device 300 also includes a lower body component 320.

The input port 312 is relatively larger in size than the output ports 340A and 34B, to allow for the input of a relatively larger volume of fluid substances. In the example shown in FIG. 13, the input port 312 is embodied as a raised soft-tip syringe press-fit connector, and the open end of a relatively large syringe can be press-fit into the input port 312. Each of the output ports 340A and 340, among possibly others not shown in FIG. 13, includes a luer lock connector. The open end of syringes can be secured onto the output ports 340A and 34B using the luer lock connectors. In alternative embodiments, the input port 312 and the output ports 340A and 340B can include other types or styles of connectors for mechanical attachment to syringes.

The device 300 also includes a fluid distribution network. The fluid distribution network is integrally formed in device 300. The fluid distribution network is formed to convey a fluid substance from the input port 312 to the output ports 340A and 340B, among other output ports. Thus, if a syringe of larger volume is coupled at the input port 312, the contents of the syringe can be expelled into and through the input port 312, into the device 300, and routed to the output ports 340A and 340B. If syringes of relatively smaller volume are coupled at the output ports 340A and 340B, the contents of the larger syringe at the input port 312 can be transferred into the smaller syringes at the output ports 340.

As also shown in FIG. 13, the device 300 includes a fluid distribution network 330. The fluid distribution network 330 is formed in a type of star configuration. The fluid distribution network 300 includes passageways or channels between the input port 312 and each of the output ports 340A and 340B. The fluid distribution network 330 includes a central distribution hub 332 and a number of fluid distribution channels 334A and 334B, among others, for conveying fluid substances from the input port 312 to the output ports 340A and 340B. The output ports 340A and 340B are in fluid communication with the distribution channels 334A and 334B, respectively. Although only two fluid distribution channels 334A and 334B are shown in FIG. 13, the fluid distribution network 330 can include additional fluid distribution channels, such as three, four, five, six, or more channels.

The central distribution hub 332 includes a channel bend 336A leading to the distribution channel 334A and a channel bend 336B leading to the distribution channel 334B. The central distribution hub 332 also includes other channel bends leading to other distribution channels, although not shown in FIG. 13. The channel bend 336A turns from the relatively vertical orientation of the input port 312 to the relative horizontal orientation of the distribution channel 334B, without a sharp transition. The channel bend 336A turns from the horizontal to the vertical orientation at a radius “R.” The radius “R” can be about one inch (e.g., +/− based on manufacturing tolerances) in one example. Other dimensions of “R,” including within a range from 0.25 to 2 inches can also be relied upon. Example dimensions include about 0.25, 0.5, 0.75, 1, 1.25, 1.5, 1.75, and 2 inches, although smaller and larger dimensions of “R” can be relied upon. The channel bends 336A and 336B can facilitate the transfer of fluid substances within the fluid distribution network 330 with less need for pressure or force to move the liquid.

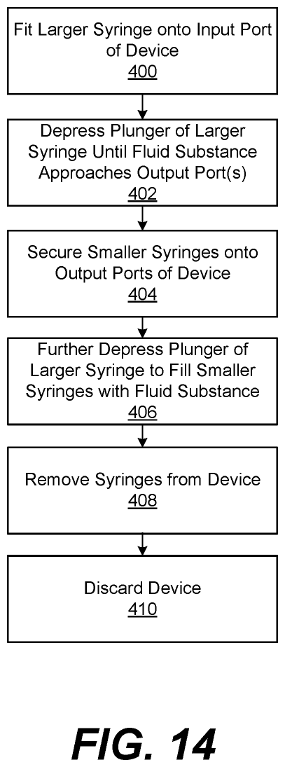

FIG. 14 illustrates an example process for a fluid splitting procedure according to various embodiments described herein. The process is described in connection with the use of the devices 10, 100, 200, and 300 described above, although other, similar devices can be used in the process. Additionally, although the process shows a certain order or sequence of steps, the steps can be varied in order. For example, step 404 can occur before step 402, and other variations are within the scope of the embodiments. Moreover, one or more of the steps shown in FIG. 14 can be omitted or skipped, such as step 410.

At step 400, the process includes fitting a larger syringe onto an input port of a device for splitting a fluid substance. The device can be any of the devices 10, 100, 200, and 300 described above, as examples, or similar devices incorporating the concepts described herein. The larger syringe can include a soft-tip or luer lock connector at one end and can be filled with a fluid substance for transfer to other, smaller syringes. An individual, such as doctor or assistant during a medical procedure, can fit the larger syringe into the input port of the device 10, 100, 200, or 300 for splitting the fluid substance at step 400. The larger syringe can hold 60 cc of the fluid substance, for example, although the process can proceed with other sizes of syringes.

At step 402, the process includes depressing a plunger of the larger syringe. The individual can depress the plunger. In turn, the fluid substance in the larger syringe will pass into the input port and the fluid distribution network of the device 10, 100, 200, or 300. The individual can continue to depress the plunger until the fluid substance approaches at least one of the output ports of the device 10, 100, 200, or 300.

With the luer lock connectors of the output ports left open (i.e., without smaller syringes being connected to them), the individual can observe the openings of the output ports of the device 10, 100, 200, or 300 at step 402, to see if the fluid substance is approaching (e.g., about to spill out of) the output ports. The individual can depress the plunger of the larger syringe until the fluid substance approaches the output ports and is about to spill out from the output ports.

At step 404, the process includes securing smaller syringes onto the output ports of the device 10, 100, 200, or 300. The individual can secure the smaller syringes onto the output ports of the device 10, 100, 200, or 300. In one example, each of the smaller syringes can be empty (i.e., evacuated of air to the extent possible), with its plunger fully depressed, and include a luer lock connector at one end. The luer lock connectors of the smaller syringes can be secured (e.g., screwed onto) onto the output ports of the device 10, 100, 200, or 300. If any of the output ports are not needed for fluid transfer, the individual can also cap, cover, or seal any unused or unconnected output ports.

At step 406, the process includes further depressing the plunger of the larger syringe to fill the smaller syringes with the fluid substance from the larger syringe. A fluid substance pushed from the larger syringe into the input port at step 406 will enter the central distribution hub and fluid distribution network of the device 10, 100, 200, or 300, spread and distribute among the fluid distribution network, and flow up and into the output ports and the smaller syringes, filling the smaller syringes.

At step 408, the process includes removing the smaller syringes from the output ports of the device 10, 100, 200, or 300. Here, the individual can unsecure (e.g., unscrew) the luer lock connectors of the smaller syringes from the output ports. At step 410, the process can include the individual discarding the device 10, 100, 200, or 300. In some cases, the device 10, 100, 200, or 300 can be cleaned and sterilized rather than being discarded at step 410.

Although embodiments have been described herein in detail, the descriptions are by way of example. The features of the embodiments described herein are representative and, in alternative embodiments, certain features and elements may be added or omitted. Additionally, modifications to aspects of the embodiments described herein may be made by those skilled in the art without departing from the spirit and scope of the present invention defined in the following claims, the scope of which are to be accorded the broadest interpretation so as to encompass modifications and equivalent structures.

1. A device for splitting a fluid substance among syringes, comprising:
an input port;
a plurality of output ports; and
a fluid distribution network for conveying the fluid substance from the input port to the plurality of output ports.

2. The device according to claim 1, further comprising:
an upper body component; and
a lower body component.

3. The device according to claim 2, wherein the input port and the plurality of output ports are integrally formed in the upper body component.

4. The device according to claim 2, wherein the fluid distribution network is integrally formed in the lower body component.

5. The device according to claim 2, wherein the upper body component and the lower body component are sealingly secured together.

6. The device according to claim 2, wherein the fluid distribution network comprises a plurality of radially-extending fluid distribution channels integrally formed in the lower body component for conveying the fluid substance from the input port to the plurality of output ports.

7. The device according to claim 6, wherein the plurality of radially-extending fluid distribution channels are formed in a star configuration.

8. The device according to claim 6, wherein each output port among the plurality of output ports is in fluid communication with a respective one of the plurality of radially-extending fluid distribution channels.

9. The device according to claim 2, wherein the fluid distribution network comprises a radially-extending fluid distribution channel and a circularly-extending fluid distribution channel integrally formed in the lower body component for conveying the fluid substance from the input port to the plurality of output ports.

10. The device according to claim 9, wherein each output port among the plurality of output ports is in fluid communication with the circularly-extending fluid distribution channel.

11. The device according to claim 1, wherein the input port comprises a press-fit connector.

12. The device according to claim 1, wherein the input port comprises a luer lock connector.

13. The device according to claim 1, wherein the input port comprises a press-fit connector with an interference-fit ledge stop.

14. The device according to claim 1, wherein the input port comprises a tapered port leading to the fluid distribution network.

15. The device according to claim 1, wherein the plurality of output ports each comprise a luer lock connector.

16. The device according to claim 1, wherein the fluid distribution network comprises a fluid channel in a series arrangement between the input port and the plurality of output ports.

17. The device according to claim 1, wherein the fluid distribution network comprises a fluid channel in a parallel arrangement between the input port and the plurality of output ports.

18. A procedure for splitting fluid substances among syringes, comprising:
fitting a larger syringe onto an input port of a device for splitting a fluid substance;
depressing a plunger of the larger syringe until the fluid substance approaches at least one of a plurality of output ports of the device;
securing smaller syringes onto the plurality of output ports of the device; and
further depressing the plunger of the larger syringe to fill the smaller syringes with the fluid substance.

19. The procedure for splitting fluid substances according to claim 18, further comprising:
removing the smaller syringes from the device; and
discarding the device.

20. The procedure for splitting fluid substances according to claim 18, wherein securing the smaller syringes comprises screwing one end of the smaller syringes onto a luer lock connector at each of the plurality of output ports of the device.

21. The procedure for splitting fluid substances according to claim 19, wherein the device comprises:
the input port;
the plurality of output ports; and
a fluid distribution network for conveying the fluid substance from the input port to the plurality of output ports.
